(19) United States
(12) Patent Application Publication
(10) Pub. No.: US 2004/0023907 A1
(43) Pub. Date: Feb. 5, 2004

(54) Infection model

(76) Inventors: Christoph Dieterich, Winnenden (DE); Steffen Rupp, Stuttgart (DE); Michaela Noll, Stuttgart (DE); Thomas Graeve, Stuttgart (DE)

Correspondence Address: Dickstein Shapiro, Morin & Oshinsky, 41st Floor, 1177 Avenue of the Americas, New York, NY, 10036-2714, US

(21) Appl. No.: 10/296,965

(22) PCT Filed: May 29, 2001
(86) PCT No.: PCT/EP01/06072

(30) Foreign Application Priority Data
May 31, 2000 (DE) 100 26 789.0
Dec. 15, 2000 (DE) 100 62 626.2

(51) Int. Cl. C12N 15/06; C12N 5/06

(52) U.S. Cl. 514/44; 435/320.1; 435/325

(57) ABSTRACT

This present invention relates to means and methods for analyzing and diagnosing infections in humans or animals caused by pathogenic and/or parasitic microorganisms, means and methods for analyzing and diagnosing degenerated or genetically engineered human or animal cells, as well as means and methods for investigating and testing anti-infective agents and medications against tumors, as well as three-dimensional in vitro organ and tissue models, in particular of tissues susceptible for infections, such as intestines, skin, cornea, trachea, and mucous membranes.

### FIELD OF THE INVENTION

The invention relates to means and methods for analyzing and diagnosing infections and/or diseases of the human or animal body caused by pathogenic and/or parasitic microorganisms, means and methods for analyzing and diagnosing degenerated human and animal cells, means and methods for analyzing and diagnosing genetically engineered human and animal cells, as well as means and methods for investigating and testing anti-infective agents and medications against tumors, in particular cytostatics, as well as three-dimensional animal in vitro organ and tissue models, in particular of tissues susceptible for infections, such as intestines, skin, cornea, trachea, and mucous membranes.

### BACKGROUND OF THE INVENTION

Macroorganisms, such as humans, can be attacked by a large number of microorganisms, including both prokaryotic organisms, such as bacteria, as well as eukaryotic organisms, such as fungi and protozoa, as well as, in a further sense, viruses. This may lead to different consequences in an affected macroorganism. An infection with microorganisms may, for example, lead to the development of an infectious disease. Other microorganisms lead a parasitic life in or on the host organism, i.e., they live by exploiting their host organism, whereby the latter does not become seriously ill. On the other hand, it is known of some viruses, especially oncogenic viruses, that they are able to neoplastically transform in vivo in human or animal cells, with some of them being associated with the development of degenerated cells and tumor pathogenesis.

The infection of a human or animal organism by a microorganism consists of several steps, such as the transfer of the microorganism to its host organism, the adhesion of the microorganism to the cells or tissues of the host organism, the entrance (invasion or penetration) of the microorganism into specific cells or tissues of the host organism, and the multiplication of the microorganism in them. An infection is determined by the infectious properties, for example, the transmissibility, contagiousness, adhesive power, penetration power, and reproduction capability as well as the pathogenic properties of the microorganism. However, the development and progression of an infectious disease is also determined by the susceptibility and immunity of the attacked host organism. The mechanisms of an infection differ greatly, depending on the specific microorganism, for example, whether the organism in question is prokaryotic or eukaryotic, and depending on the affected host organism, for example, which organs or tissues are affected.

The development and progression of a tumor disease also depend, in addition to the properties of the virus, on the susceptibility and immunity of the affected host organism. It is, for example, known that viruses preferably cause the formation of neoplasms in immunoincompetent organisms.

In accordance with the previous, the medications used for treating either infectious diseases or tumors act in different ways. Anti-infective agents used for infectious diseases, which damage or kill the microorganisms in the body of the affected organism, aim for an inhibition of the cell wall synthesis of the microorganism, an interference in the permeability of its cytoplasm membrane, a blockade of its protein synthesis, and/or a suppression of its nucleic acid synthesis, without damaging the host organism itself. However, in cancer tumors caused by oncogenic viruses, the actual causative agent, i.e., the oncogenic virus, cannot be fought in a targeted manner at this time; rather, the degenerated cell is destroyed or its growth is inhibited, for example, by using cytostatics.

In the past, primarily animal experiments have been used to study the complex interactions between a human or animal host and a microorganism leading to the development of an infectious diseases, as well as for analyzing the complex cellular and/or viral mechanisms leading to the development of degenerated animal cells. For the most part, animal experiments were also used to develop and test anti-infective agents or cytostatics, for example, within the context of preclinical tests. It was found, however, that the results obtained from animals only can be transferred to humans to a limited extent.

Along with the development of cell culture techniques, experiments with two-dimensional in-vitro cell systems for supplementing or replacing animal experiments were also performed. For example, studies with the human pathogen candida albicans fungus that causes candida mycoses in humans and may lead to life-threatening infections in immunosuppressed patients (Mitchell, Curr. Opin. Microbiol., 1 (1998), 687-692) were also performed on such cell systems. Among other things, an epidermis model constructed from one type of cell and consisting of a cell monolayer was developed in order to study the adhesion and penetration of candida (Korting et al., J. Infect., 36 (1998), 259-267; Zink et al., Infect. Immun., 64 (1996), 5085-5091).

However, the disadvantage of such two-dimensional in vitro cell systems is that they provide no statements regarding a further infection mechanism or the exact progression of tumor pathogenesis. As a result of their structure, these systems do not allow any interactions between different cell types, as it is the case, for example, in vivo in complete organs. These systems also do not contain any connective-tissue-specific matrix but are based on synthetic membranes. These systems, for example, in the case of an infectious disease, therefore do not allow a more detailed analysis of the complex interactions between a pathogen, the cell types making up the organs or tissues, and the solid connective tissue matrix. This means that such systems can only be used to a very limited extent for developing and testing anti-infective agents and cytostatics.

### BRIEF SUMMARY OF THE INVENTION

The technical objective underlying this invention therefore is to develop means and methods for analyzing and diagnosing infections in human or animal host organisms caused by pathogenic or parasitic microorganisms, means and methods for analyzing and diagnosing degenerated or genetically engineered human or animal cells, as well as means and methods for studying and testing diagnostics and therapeutics, especially anti-infective agents and medications for treating tumors, especially cytostatics, that overcome the previously described disadvantages of the state of the art and are in particular suitable for studying the interactions between pathogenic and parasitic microorganisms and their target organs or tissues that lead to infections as well as the cellular and/or viral mechanisms leading to the formation of tumors, and which therefore allow the development and testing of specific diagnostics and therapeutics, such as anti-infective agents or cytostatics.

The invention solves its underlying technical objective by providing in vitro methods for differentiating and/or multiplying isolated animal and human cells, in the course of which three-dimensional animal or human in vitro organ or tissue models are prepared. According to the invention, especially primary cells or other cells from tissues or organs susceptible to infections can be used, such as intestines, skin, cornea, trachea, or mucous membranes, but also degenerated cells to be tested, especially of the previously mentioned organs or tissues, or genetically engineered cells to be tested, especially of the previously mentioned organs and tissues. Based on this, and utilizing the previously mentioned methods, the present invention makes available in vitro methods for co-cultivating the differentiated and/or multiplied cells with pathogenic and parasitic microorganisms and in vitro methods for studying the interaction of substances with the in vitro co-cultivated, differentiated and/or multiplied cells and microorganisms. These methods permit the analysis of infection progressions and make available diagnostics and therapeutics useful in the area of infection medicine.

The results achieved with the organ test systems according to the invention may be more meaningful than the results obtained with animal experiments and may ensure better transferability to humans.

According to the invention, the previously mentioned cells are cultivated in a three-dimensional, gel-like, connective-tissue-like biomatrix, in which they are able to multiply. In addition to the cells to be cultivated, this biomatrix contains a collagen network constituted from a collagen solution, i.e., tissue-specific matrix proteins. Depending on the desired type of tissue, other cell types, preferably other primary cells, can be applied to this cell-containing collagen gel. By using specific culture conditions and specific culture media, the cells contained in the biomatrix and the other cell types applied to the biomatrix may undergo a differentiation into a multilayer, three-dimensional animal tissue or organ test model.

This means that, with the help of the methods according to the invention, three-dimensional animal or human tissue and organ test models are obtained, which consist of several tissue-specific cell layers and correspond both histologically as well as functionally substantially to the native organs and tissues. These organ or tissue test models are therefore suited much better for a true to nature modeling of infection progressions in animals and humans than the standard in vitro systems constructed of only one type of cell and thus can be used for a targeted analysis of the infection and resistance mechanisms of bacteria, fungi, viruses and protozoa. This means that according to the invention the in vitro organ or tissue test models according to the invention can be inoculated with a parasitic or pathogenic microorganism and cultivated together with it under suitable conditions. The use of different primary cells from different organs for constructing the animal in vitro organ or tissue test systems also makes it possible to study the behavior of a causative agent on several tissue systems.

The invented co-cultivation of the animal in vitro tissue or organ test system according to the invention with a parasitic or pathogenic microorganism offers the possibility of studying both the infection process itself as well as the defense reaction of the corresponding, organoid cell systems. For example, larger amounts of an infected cell material and the pathogen itself can be obtained. The obtained material then can be further analyzed using standard histological, biochemical, molecular-biological or immunological methods in order to study in more detail, for example, morphological changes of infected cells, the secretion of specific substances by the pathogen, such as toxins or proteins relevant for any occurring resistance, or the secretion of specific substances by the affected cells, such as interleukins, as a defense reaction, or in order to create transcription and/or expression profiles on the basis of which, for example, virulence factors can be identified as targets for developing anti-infective agents.

The previously mentioned method according to the invention in an advantageous manner permits the screening and the analysis of potential diagnostics, with the help of which, for example, the presence of certain symptoms of an infection can be demonstrated. The invention therefore also relates to methods for identifying diagnostics or the analysis of their specificity, whereby, within the context of the invented co-cultivation of the single- or multilayer tissue or organ test systems according to the invention with infective agents, a potential or investigated diagnostic is tested for its ability to identify infections or infection processes. Within the context of these methods, the investigated diagnostics may be added to the system according to the invention, whereby it can be determined with the help of morphological, physiological or other parameters, to what extent a correlation exists between the infection condition and the marking or detection by the diagnostic.

By using the animal and human in vitro tissue or organ test systems according to the invention and the co-cultivation method according to the invention, it is possible to analyze especially the efficacy with respect to the mechanism of action and/or the side effects of therapeutics, such as anti-infective agents, much more accurately than with standard test systems, for example, with respect to their effects on gene expression, metabolism, proliferation, differentiation, and reorganization of cells of an in vitro organ or tissue test system. These tests of active substances, therapeutics, and diagnostics as well as of the interactions between infectious agent and cultivated cells in animal and human in vitro tissue test systems according to the invention may include both standard morphological or histological methods as well as standard biochemical, toxicological, immunological and/or molecular-biological methods.

According to the invention, the methods according to the invention and the means employed in them, i.e., the mono- and multilayer in vitro tissue or organ systems may be used for screening potential active substances and for studying the properties, such as specificities, as well as interactions of active substances with target cells. According to the invention, the term “active substance” stands for any substance, but also for other agents, including physical influences, such as electromagnetic radiation, radioactivity, heat, sound, etc., which are able to influence or identify biological cells or parts thereof, especially cell organelles. In particular, such active substances may be of a chemical nature, for example diagnostics or therapeutics. In connection with the invention at hand, the term “diagnostics” stands for any substances that are able to identify specifically the presence or absence of conditions, processes, or substances, and in particular are able to provide conclusions about pathological conditions. Diagnostics often have identifying and marking functions. The term “therapeutics” in particular stands for such substances that can be used either prophylactically or concomitant with a disease in order to prevent, alleviate or eliminate pathological conditions. Within the context of this invention, the term “diseases” also includes conditions such as unnatural emotional conditions, pregnancies, symptoms of aging, developmental disorders, etc. Within the context of this invention, the term “therapeutics” also includes substances that exclusively or additionally also have cosmetic effects.

The methods according to the invention also are suitable for studying the mechanisms of tumor-pathogenesis and/or for studying substances for their suitability as a medication, for example, against a specific tumor. For example, an in vitro organ or tissue test system constructed of degenerated cells, in particular of the previously mentioned organs or tissues, can be used to obtain larger amounts of a degenerated cell material. The obtained material then can be further analyzed using standard methods, for example, histological, biochemical, molecular-biological or immunological methods in order to study, for example, morphological changes of degenerated cells or the secretion of specific substances in more detail or to create transcription and/or expression profiles. Furthermore, the effect of medications or other substances with respect to their ability of inhibiting cell division also can be studied with an in vitro organ or tissue test system constructed of degenerated cells. On the other hand, an in vitro organ or tissue test system constructed of non-degenerated primary cells can be co-cultivated with oncogenic viruses. This method can be used to study the multiplication and/or distribution of oncogenic viruses in the cells of the in vitro test system in the presence or absence of specific substances that are able to inhibit specific functions of the virus.

The methods according to the invention in addition also can be used to study cells, especially of the previously mentioned genetically engineered tissues and organs. It is, for example, possible to test genetically engineered cells with respect to gene therapy for eliminating gene-specific malfunctions or for the restoration of normal gene function in diseases of the previously mentioned organs.

A preferred embodiment of the invention comprises the cultivation of animal or human cells in a three-dimensional, gel-like biomatrix for the multiplication of these cells and for preparing a three-dimensional animal or human in vitro organ or tissue test system.

In an especially preferred embodiment, the invention comprises the cultivation of human dermal fibroblasts in the biomatrix for preparing a three-dimensional human in vitro skin equivalent consisting of a dermis equivalent and an epidermis equivalent.

In connection with the present invention, the term “cultivation of cells” means a preferably in vitro maintenance of the life functions of cells, in particular of fibroblasts, in a suitable environment, for example, by adding and removing metabolic educts and products, in particular for the multiplication of cells. In connection with the present invention, the term “dermal fibroblasts” means naturally occurring fibroblasts, especially occurring in the dermis, or genetically engineered fibroblasts or their precursors. Fibroblasts are the precursors of dermal fibrocytes. The fibroblasts may be of animal or human origin.

The biomatrix intended for cultivating the fibroblasts contains the fibroblasts to be cultivated as well as a collagen network, newly constituted from a preferably fresh collagen solution, with a concentration of preferably 3.5 to 4.5 mg of collagen per ml of biomatrix. The collagen network is obtained from a preferably cell-free, acidic solution of collagen I, whereby the protein concentration of the collagen solution is preferably 5 to 7 mg/ml. The pH value of the collagen solution is preferably 3.8. In order to prepare the fibroblast-containing biomatrix according to the invention, a solution containing a preferably 5× concentrated cell culture medium, buffer, preferably HEPES buffer, serum, preferably fetal calf serum (FCS) and chondroitin-(4/6)-sulfate, and preferably 1.5×10⁵/ml fibroblasts, in particular precultivated fibroblasts, is added to the collagen solution, preferably at 4° C., and is well mixed. This mixture is gelled by increasing the temperature to room temperature or 37°. After gelling the gels, fibronectin is placed on top of the gels. Fibronectin functions in vivo by binding to other macromolecules, for example collagen, and by binding cells to neighboring cells. The following cultivation of the fibroblasts in the collagen gel preferably takes place in submerse culture. In connection with the present invention, a “submerse culture” means a method for cultivating cells in which the cells are covered with a nutrient solution. The biomatrix containing fibroblasts thus is coated with a cell culture medium and incubated at 37° C.

One to three, preferably two days after the previously described incubation of the gels, keratinocytes are seeded onto the gel. In connection with the present invention, “keratinocytes” mean cells of the epidermis that form keratinized squamous epithelium or genetically engineered keratinocytes or their precursors, which may be of animal or human origin. The keratinocytes seeded on the collagen gel are preferably, if possible, precultivated, undifferentiated keratinocyte stem cells from human biopsy tissue, i.e. cytokeratin 19- or integrin ∃1-positive, basal stem cells. The seeding of the keratinocytes on the biomatrix takes place in a cell culture medium, preferably in KBM medium (Clonetics) that contains 5% fetal calf serum. The biomatrix is then coated with KBM medium containing human epidermal growth factor (0.1 μg/500 ml medium) (hEGF), BPE (beef hypophysis extract) (15 mg protein/500 ml medium) and 0.8 mM CaCl₂ and undergoes a 1- to 3-day submerse cultivation. A complete differentiation of the keratinocyte layers is achieved with an airlift culture with KBM medium containing 1.8 mM CaCl₂ and without hEGF and BPE. In connection with the present invention, an “airlift culture” means a culture in which the height of the nutrient medium level is exactly adapted to the height of the biomatrix, while the keratinocytes or the cell layers formed by the keratinocytes are above the nutrient medium level and are not covered by the nutrient medium, i.e. the cultivation takes place at the air/nutrient medium interface, whereby the cultures are supplied with nutrients from the bottom. After a preferably 12- to 14-day airlift culture, a skin-specific in vitro full skin model, consisting of dermis equivalent and epidermis equivalent and which can be used advantageously for the test methods according to the invention, develops.

The invention therefore also relates to a skin-specific in vitro full skin test model, in particular animal or human in vitro full skin test model, that was prepared in accordance with the method according to the invention and that comprises at least 2 to 4 proliferative, several differentiating, and at least 4 to 5 keratinized cell layers, whereby the epidermis equivalent comprises the stratum basale, stratum spinosum, stratum granulosum, and stratum corneum, and whereby a functional basal membrane of matrix proteins is contained between the dermis equivalent and the epidermis equivalent. This model is suited very well as a test system for studying potential or actual active substances, such as therapeutics, diagnostics, or for investigations regarding the progression of infection processes.

Another particularly preferred embodiment of the invention comprises the cultivation of intestinal fibroblasts in the biomatrix for preparing a three-dimensional, human in vitro intestinal test system consisting preferably of Caco2 cells or intestinal epithelial cells or other human cell lines.

In connection with the present invention, the term intestinal fibroblasts means naturally occurring fibroblasts, especially occurring in the intestinal tissue, or genetically engineered fibroblasts or their precursors. The intestinal fibroblasts may be of animal or human origin.

In connection with the present invention, the term intestinal epithelial cells means naturally occurring epithelial cells, especially occurring in the intestinal tissue, or genetically engineered epithelial cells or their precursors. The intestinal epithelial cells may be of animal or human origin.

To prepare the intestinal fibroblast-containing biomatrix according to the invention, a solution, also called a gel solution, containing a preferably 2× concentrated cell culture medium, buffer, preferably HEPES buffer, and serum, preferably 10% serum, and preferably 1.5×105/ml intestinal fibroblasts, in particular precultivated intestinal fibroblasts, are added to the collagen solution in a volume ratio of 1:1, preferably at 4° C., and is well mixed. If an x times concentrated gel solution is present, the collagen solution is preferably mixed in a volume ratio (x−1):1 with the gel solution, whereby x is the concentration factor. This mixture is gelled by increasing the temperature to room temperature or 37°. The following cultivation of the intestinal fibroblasts in the collagen gel preferably takes place in submerse culture. The biomatrix containing fibroblasts is incubated at 37° C.

The intestinal epithelial cells are preferably seeded onto the gel 1 to 3 days following incubation of the gels.

If possible, the intestinal epithelial cells seeded on the collagen gel are preferably precultivated, undifferentiated intestinal epithelial cells. The seeding of the intestinal epithelial cells onto the biomatrix takes place in a cell culture medium, preferably in DMEM medium (Dulbecco's Modified Eagle Medium, Life Technologies, Cat. No. 41966 or 52100) that contains 10% FCS and glutamine (2 mM) and 1% non-essential amino acids (MEM, Life Technologies, Cat. No. 11140). The biomatrix is coated with DMEM medium containing 10% FCS and glutamine (2 mM) and 1% non-essential amino acids and undergoes a 10- to 20-day submerse cultivation, until a complete differentiation of the epithelial layer or epithelial layers occurs that can be advantageously used for the test methods according to the invention.

Another advantageous embodiment of the invention comprises the co-cultivation of a three-dimensional in vitro organ or tissue test system prepared according to the invention with a pathogenic or parasitic microorganism. In connection with the present invention, the term “pathogenic or parasitic microorganisms,” also called infectious agents here, stands for both eukaryotic as well as prokaryotic microorganisms, such as bacteria, fungi, protozoa, viroids, but also prions or viruses that attack a macroorganism, in particular a human or animal organism, and are able to live in or on the tissues of this organism and may, but do not necessarily have to, lead to an infection of this organism. In connection with the invention, the term “co-cultivation” stands for a preferably in vitro, simultaneous maintenance of the life functions of animal cells and microorganisms in the same environment that is suitable for both, for example with addition and removal of metabolic educts and products, especially also a simultaneous multiplication of the cells and microorganisms.

In a preferred embodiment, a co-cultivation of the human-pathogenic candida albicans fungus is performed with the human in vitro skin test system prepared according to the invention in order to investigate the infection process of candida in human skin tissue, and with the human in vitro intestinal test system prepared according to the invention in order to investigate the infection process of candida in human intestinal tissue. The results achieved with candida, especially the detailed description of the infection process, also can be transferred to other pathogens.

In an especially preferred embodiment, the present invention relates to the co-cultivation of the human-pathological microorganism candida albicans with the human in vitro skin test system or the human in vitro intestinal test system in order to investigate the first stage of the infection process, i.e. the adhesion of the pathogen to the skin or intestinal cells. The adhesion of the pathogen is investigated using the virulent candida strain Sc5314 and the avirulent candida strain Can34 that have already been studied in a mouse macrophage model (Longitudinal et al., Cell, 90 (1997), 939-949). The in vitro skin test system or the in vitro intestinal test system is in each case inoculated in submerse culture with about 10³ pathogenic organisms and cultivated with shaking. At defined times, for example every 30 minutes (up to a maximum of 4 hours), aliquot amounts are removed and plated out into Petri dishes with appropriate nutrient media, for example YPD full medium (Difco). After an appropriate incubation time, the number of colonies in the Petri dishes is determined. The adhesion of the pathogens on the in vitro organ test systems can be determined based on the comparison between the determined colony count and the originally inoculated number of pathogens. With the help of this method, it could be demonstrated in this way that the virulent strain has the ability to adhere both to skin and to intestinal cells, while only a slight adhesion could be demonstrated for the avirulent stem.

In another particularly preferred embodiment, the present invention relates to the co-cultivation of the human-pathogenic microorganism candida albicans with the human in vitro skin test system prepared according to the invention or with the human in vitro intestinal test prepared according to the invention in order to investigate another stage of the infection process, i.e. the penetration/invasion of the pathogen into cells. For this, the organoid tissue test systems are co-cultivated using the airlift process with the previously described avirulent and virulent pathogen strains. The pathogen is preferably fixed with a cell count of 10³/ml in 1% agar, and agar pieces with a diameter of 4 mm are placed onto the organoid tissue test systems for up to 98 hours. The penetration of the pathogen into the organoid structures was studied after 16 hours, 24 hours, 72 hours, 86 hours, and 98 hours using histological methods on thin sections, employing the PAS staining method (McManaus, Romeis, 17th ed., page 393). Using the histological sections, the invasion process of the virulent candida strain into the deeper layers of the connective-tissue-like matrix can be documented.

Another advantageous design of the invention provides for the investigation of the effect of chemical substances, in particular anti-infective agents, or of agents on the infection process or the growth of a pathogenic microorganism by using an in vitro organ or tissue test system prepared according to the invention and the co-cultivation process according to the invention. In connection with the invention, the term “agent” particularly stands for chemical, biological or physical means, such as light or heat, which are able to exert a potential effect on living cells. The studies regarding the effect of anti-infective agents also were performed with candida. Currently there exist two substance classes, i.e. azoles and polyenes, that are preferably used as antimycotics against systemic infections. Both classes of substances have disadvantages. Polyenes have severe side effects, and more and more resistance develops against azoles (DiDomenico, Curr. Opin. Microbiol., 2 (1999), 509-515; Georgopapadakou, Curr. Opin. Microbiol., 1 (1999), 547-557). For this reason, a specific development of new antimycotics is urgently needed.

In a preferred embodiment, the method for investigating the adhesion of candida on the in vitro intestinal test system according to the invention and on the in vitro skin test system according to the invention is modified in such a way that the co-cultivation preparation contains an antimycotic, in particular amphothericin B or fluconazole. By using this method, it could be demonstrated that both antimycotics had no influence on the adhesion of the pathogen, but rather influenced its growth rates.

In another preferred embodiment, the method for investigating the penetration/invasion of candida with the in vitro intestinal test system and the in vitro skin test system was modified in such a way that the co-cultivation preparation contained amphothericin B or fluconazole. With the help if this method it could be demonstrated that the invasion of the virulent pathogen strain could only be prevented with a complete inhibition of growth, whereby according to the invention also new active substances were tested.

An especially preferred embodiment of the invention relates to the analysis of degenerated cells. In connection with the invention, the term “degenerated” includes all changes of a normal cell, for example cell polymorphism, anisocytosis, nuclear polymorphism, polychromasia, abnormal nucleus-plasma relation, and aneuploidy, which may lead to an abnormal differentiation or dedifferentiation and a deregulated growth of the cell, and in particular also relates to the cells of malignant tumors. From degenerated cells, in particular from the previously mentioned organs and cells, an in vitro organ or tissue test system is constructed in order to obtain larger amounts of the degenerated cell material. The obtained material is further analyzed using standard methods, for example histological, biochemical, molecular-biological, or immunological methods in order to study the secretion of specific substances and create transcription and expression profiles. The effect of medications and substances potentially suitable as medications, in particular with respect to their ability of inhibiting cell division, is studied in the in vitro organ or tissue test system constructed of degenerated cells.

In an especially preferred embodiment of the invention, patient-specific degenerated cells are used to establish an in vitro organ test system in order to study therapeutic options for the specific tumor disease of the patient.

Another preferred embodiment of the invention provides for the study of genetically engineered cells, especially of the previously mentioned tissues and organs. In connection with the present invention, the term “genetically engineered cells” includes all cells that have been manipulated using genetic engineering processes, whereby either foreign DNA was introduced into the cell, or its own DNA was modified, for example by deletions, conversions, and insertions. An especially preferred embodiment provides for the in vitro testing of genetically engineered cells, in particular for their functionality, with respect to a gene therapy of patient-specific diseases, whereby an in vitro organ test system is established by using such genetically engineered cells.

The invention also relates to a preferably gel-like biomatrix in which the previously mentioned cultivation procedures can be performed, i.e. a biomatrix with cells of a tissue type.

The combination of biomatrix and the cells cultivated therein according to the invention may be used, as previously described, for preparing an in vitro organ or tissue test system.

The term biomatrix stands for a gel structure that contains collagen, cell culture medium, serum, and buffer, preferably HEPES buffer. The collagen solution used for preparing the biomatrix is a solution with a high content of non-denatured, native collagen in an acidic, aqueous medium, preferably with a pH value of 3.8, for example in acetic acid, preferably in 0.1% acetic acid solution. A high content of non-denatured collagen means a total collagen content in the solution of ≧50%, in particular ≧60%, ≧70%, ≧80%, ≧90%, or ≧95%, preferably ≧99%. In a preferred embodiment, no lyophilized collagen is used for this. The collagen content of the solution is preferably 3 mg of collagen per ml of solution to 8 mg of collagen per ml of solution, more preferably 5 mg of collagen per ml of solution to 7 mg of collagen per ml of solution, most preferably 6 mg of collagen per ml solution. It is preferred that collagen is used for this, which, after extraction, for example from rat tails, is incubated in 0.1% acetic acid for three to fourteen days at 4° C. with stirring, and whereby undissolved collagen parts is centrifuged off. Preferred cell culture media are DMEM (Dulbecco's Modified Eagle Medium) and M199. However, it is possible to use any desired cell culture medium that permits the cultivation of cells. It is preferred that for the serum fetal calf serum (FCS) or human autologous serum is used, and for the buffer, for example, HEPES buffer. The pH value of the solution of cell culture medium, buffer, and serum in the preferred embodiment is 7.5 to 8.5, for example 7.6 to 8.2, in particular 7.8. Naturally, the biomatrix may contain further factors, for example growth factors, adhesives, antibiotics, selection agents, etc.

The invention therefore also relates to methods for preparing a biomatrix that contains cells, whereby in a first step fresh collagen, for example from rat tails, is prepared by collecting collagen fibers extracted from collagen-containing tissue in buffer solution, superficially disinfecting them in alcohol, and then washing them in buffer solution and transferring them into an acidic solution with a pH value of 0.1 to 6.9, preferably 2.0 to 5.0, especially preferably 3.0 to 4.0, in particular 3.3, for example a 0.1% acetic acid solution. In a further step, the collagen in the solution is stirred at 2 to 10° C., in particular 4° C., for several days, for example for 3 to 14 days, the undissolved collagen parts are centrifuged off, and a collagen solution with a collagen content of 3 mg/ml to 8 mg/ml is stored at 2 to 10° C., for example at 4° C. It is naturally also possible to temporarily store the solution in a frozen state, for example at −10° C. to −80° C., in particular at −20° C. To prepare the biomatrix containing the cells according to the invention, a solution, also called a gel solution, consisting of a preferably several times (x times) concentrated cell culture medium, serum, and buffer, is mixed in a third step with precultivated and centrifuged-off cells, using preferably 1×10⁵ to 2×10⁵ cells per ml, preferably 1.5×105 cells per ml. This solution, or suspension, with a pH value from 7.5 to 8.5, preferably 7.6 to 8.2, in particular 7.8, is then mixed, for example in a ratio of 1:2, with the previously mentioned collagen solution at 2 to 10° C., in particular at 4° C. The mixing ratio (volume) of collagen solution to gel solution (buffer, serum, cells, culture medium) is preferably 1:1, whereby for an x times concentrated gel solution a volume ratio of (x−1):1 collagen solution to gel solution is preferred. The gel solution is then pipetted into culture containers and is coated, after gelling at 37° C., with medium. The biomatrix is then cultivated for at least two days, after which time cells of other tissue types, for example also immune system cells, can be applied to it.

Other advantageous embodiments of the invention are derived from the specification.

The invention is described in more detail using the following figures and examples.

### BRIEF DESCRIPTION OF THE DRAWINGS

FIG. 1 shows a longitudinal section of the in vitro intestinal system prepared according to the invention.

FIG. 2 shows a longitudinal section of the in vitro intestinal system prepared according to the invention with adhering cells of the candida albicans fungus (adhesion of a virulent strain).

FIG. 3 shows a longitudinal section of the in vitro intestinal system prepared according to the invention with penetrated cells of the candida albicans fungus (invasion of a virulent strain).

### DETAILED DESCRIPTION OF THE INVENTION

### EXAMPLE 1

Preparation of a Three-Dimensional Human in vitro Skin Test System Preparation of a Gel Solution

20 parts of 5× concentrated M 199 cell culture medium (Life Technologies, 1999, Cat. No. 42966 or 52100; DMEM), 10 parts of HEPES buffer (4.76 g in 100 ml PBS solution, pH value 7.3), and 1 part chondroitin. (4,6) sulfate (5 mg/ml in PBS) are mixed, and the pH value of the mixture is adjusted to 7.8. The mixture is sterilized by filtration, after which 10 parts of fetal calf serum are added.

Preparation of a Collagen Solution

Collagen-containing tissue, for example tendons from rat tails, is used to prepare a collagen solution. All work is performed under sterile conditions with sterile materials. After storage at −20° C., the rat tails are superficially disinfected with 70% alcohol. The rat tails are skinned, and the individual collagen fibers are extracted. If using other starting tissues, any possibly present cells may be carefully removed with a mechanical, enzymatic, or chemical treatment. The collagen fibers are collected in phosphate-buffered saline (PBS) (pH 7.2), superficially disinfected in 70% alcohol for 10 minutes, and then washed thoroughly with PBS. The weight of the fibers is determined, and the fibers are transferred into a 0.1% acetic acid solution (final concentration of approximately 8 to 12 mg/ml). This preparation is stirred for a period of about 3 to 14 days at 4° C., and any undissolved collagen parts are then removed using centrifugation (1,000 rpm, 1 hour, 8° C.). As a result, the collagen is dissolved, and is no longer in fiber, network or matrix form.

Preparation of the Collagen Gels Containing the Dermal Fibroblasts (Preparation for 24 Inserts)

16 ml of collagen solution are added into a 50 ml centrifuge tube and placed on ice. Precultivated, dermal fibroblasts are harvested and counted. 1.2×10⁶ fibroblasts are placed into 8 ml of ice-cold gel solution, are well suspended, and added to the collagen solution without any air bubbles. Gel solution and fibroblasts are well mixed. 600 μl each of the mixture are carefully poured into the well of a 24-well microtiter plate (diameter of 10 mm per well). The mixture is gelled by a two-minute incubation at 37° C. After gelling the mixture, 50 μl each of fibronectin (5 μg/ml) are placed on each insert. Following a 10 minute incubation at 37° C. or a 30 minute incubation at room temperature, 1 ml of M199 medium is added for each well, whereby the inserts are coated with the medium. The fibroblasts contained in the gel undergo this submerse cultivation for 1 to 2 days at 37° C., whereby the medium is replaced with fresh medium every 12 hours.

Seeding of the Keratinocytes and Cultivation of the in vitro Skin Test System

Prior to seeding the keratinocytes, the medium is first carefully aspirated from the wells of the microtiter plate and from the gels. Then 500 μl of KBM medium (Clonetics) that contains 5% FCS is added for each well. The gels are coated with 50 μl of fibronectin solution each and are incubated for 1 hour at 37° C. Then 100,000 keratinocytes in 50-100 μl of KBM medium that contains 5% FCS are seeded for each gel and are incubated for 1 to 2 hours at 37° C. Then 500 μl KBM medium that contains 5% FCS, 8 mM CaCl₂, hEGF (0.1 μg/500 ml medium) and BPE (15 mg/500 ml medium) is added, and the gels undergo a submerse cultivation for 1 to 3 days, whereby the medium is replaced daily with fresh medium. The gels then each undergo another submerse cultivation for another 2 to 3 days in 1 to 1.5 ml of KBM medium that contains 2% FCS, 8 mM CaCl₂, HEGF (0.1 μg/500 ml medium) and BPE (15 mg/500 ml medium). The gels then undergo an airlift cultivation with the developing skin equivalent. For this purpose, the gels are transferred to a plate with 6 wells, and 1.5 to 2 ml of KBM medium with a CaCl₂ content of 1.88 mM, without hEGF and BPE, are added for each well, whereby the level of the medium is exactly adapted to the height of the gel, while the keratinocytes or the layers formed by the keratinocytes are not covered by the medium. The airlift cultivation is continued for at least 12 to 14 days.

### EXAMPLE 2

Providing a Three-Dimensional Human in vitro Intestinal Test System Preparation of a Gel Solution

77.5 parts of 2× concentrated DMEM cell culture medium (Life technologies, Cat. No. 41966 or 52100, 1999), 20 parts of FCS, 2.5 parts of HEPES buffer (71.49 g in 100 ml PBS solution, pH value 7.8) are mixed, and the pH value of the mixture is adjusted to 7.4. The mixture is sterilized by filtration.

Preparation of the Collagen Gels Containing the Fibroblasts (Preparation for 24 Inserts)

7.5 ml of collagen solution are added into a 50 ml centrifuge tube and placed on ice. Precultivated fibroblasts are harvested and counted. 1.2×10⁶ fibroblasts are placed into 7.5 ml of ice-cold gel solution, are well suspended, and added to the collagen solution without any air bubbles. Collagen solution and gel solution with fibroblasts are well mixed. 300 μl each of the mixture are carefully poured into the well of an insert. The inserts are located in a microtiter plate with 24 wells. The mixture is gelled by a two-minute incubation at 37° C. After gelling the mixture, 1 μl each of medium is placed on and next to each insert. The fibroblasts contained in the gel undergo this submerse cultivation for 1 to 3 days at 37° C., whereby the medium is replaced with fresh medium every 48 hours.

Seeding of the Intestinal Epithelial Cells and Cultivation of the Intestinal Equivalent

Prior to seeding the Caco2 cells, the medium is first carefully aspirated from the wells of the microtiter plate and from the gels. For each gel, 200,000 epithelial cells in 200 μl DMEM (see above), containing 10% FCS are then seeded, 600 μl of medium is placed next to the inserts, and cultivation is performed for 10 to 20 days at 37° C. The medium is replaced every 48 hours.

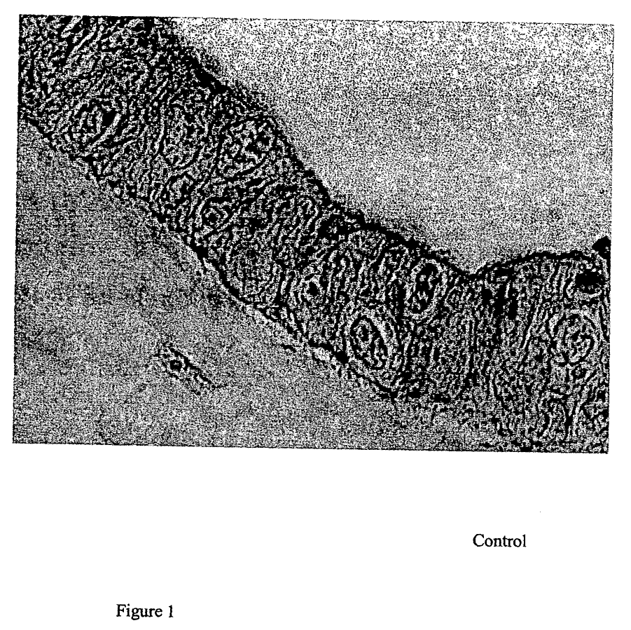

An intestinal equivalent prepared in this manner is shown in FIG. 1.

### EXAMPLE 3

Co-Cultivation of candida albicans with the in vitro Skin Test System or the in vitro Intestinal Test System for Determining the Adhesion of the Pathogen on the Cells

In order to determine the adhesion of the pathogen on cells, 12 each inserts of the human in vitro skin test system held in submerse culture or 12 each inserts of the in vitro intestinal test system held in submerse culture were infected each with 10³ pathogenic organisms of the virulent candida strain Sc5314 or of the avirulent candida strain Can34 (Longitudinal et al., Cell 90 (1997), 937-949). The inserts were then incubated with shaking at 37° C. for 30, 60, 90, 120, 150, or 180 min.

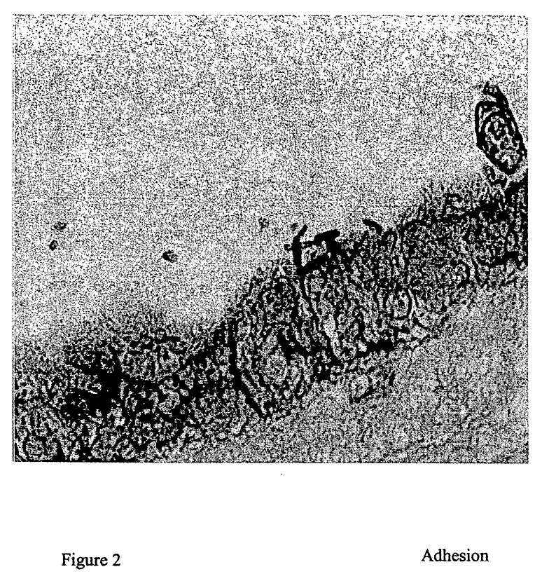

At the specified times, the supernatant was removed and plated out in Petri dishes with YPD nutrient medium. After an incubation period of 2 days, the colonies in the Petri dishes were counted. Based on the comparison between the determined colony count and originally inoculated number of pathogens, it was determined that approximately 95% of the virulent strain and 10% of the avirulent strain adhered to the in vitro skin model after 2 hours. In the in vitro intestinal model, adhesion was demonstrated for about 95% of the virulent strain (FIG. 2) and 10% of the avirulent strain.

### EXAMPLE 4

Co-Cultivation of candida albicans with the in vitro Skin Test System or the in vitro Intestinal Test System for Determining the Penetration of Cells by the Pathogen

In order to determine the penetration of the pathogen, 12 inserts of the in vitro skin system held in airlift culture and 12 inserts of the in vitro intestinal system held in airlift culture each were infected with 10³ pathogenic organisms of the virulent candida strain Sc5314 or the avirulent candida strain Can34. The inserts then were incubated up to 3 days at 37° C.

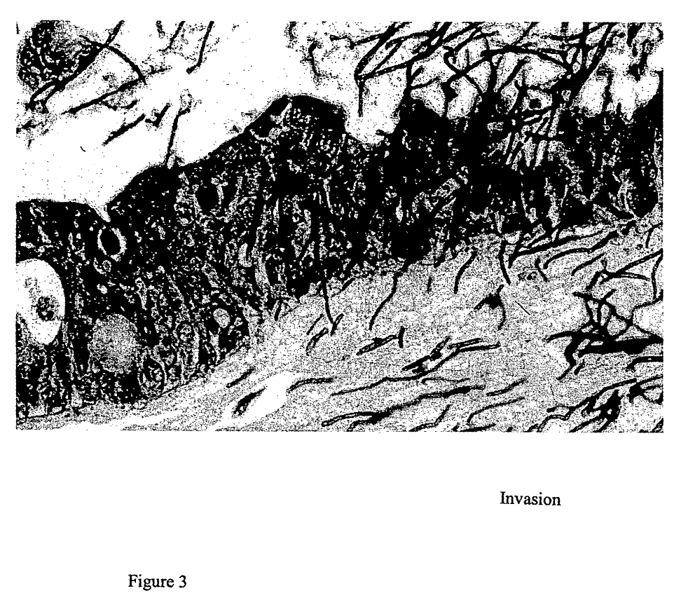

The penetration of the pathogen into the organoid structures was investigated after about 18 to 24 hours using histological methods on thin sections, employing the PAS staining method both for the in vitro skin system as well as for the in vitro intestinal system. Using the histological sections, the invasion process of the virulent candida strain into the deeper layers of the connective-tissue-like matrix was documented (FIG. 3), demonstrating that the virulent candida strain spreads in star shape from the infection site into the connective tissue, whereas the avirulent strain was not able to penetrate the epithelial cells and also did not show any adhesion.

### EXAMPLE 5

Influence of Antimycotics on the Adhesion of candida albicans to Skin and Intestinal Cells in vitro

In order to determine the influence of antimycotics on the adhesion of candida albicans on skin and intestinal cells, 12 inserts of the in vitro skin test system held in submerse culture and 12 inserts of the in vitro intestinal test system each were infected with 10³ pathogenic organisms of the virulent candida strain Sc5314 or the avirulent candida strain Can34. Amphotericin B was added to 5 inserts in a concentration of 0.1, 0.5, 1.0, and 2.0 μg/μl, and fluconazole was added to 5 inserts in a concentration of 0.1, 0.5, 1.0, and 2.0 μg/μl. The inserts were then incubated with shaking at 37° C. for up to 3 days.

After 16, 24, 72, 86, and 98 hours, aliquot amounts were removed and plated out in Petri dishes with YPD nutrient media. After an incubation period of 2 days, the colonies in the Petri dishes were counted. Based on the comparison between the determined colony count of the samples without addition of an antimycotic and the colony count for samples with addition of an antimycotic it was determined that the adhesion of the virulent candida strain could only be prevented by adding amphotericin B and fluconazole and by an inhibition of growth.

### EXAMPLE 6

Co-Cultivation of candida albicans with the in vitro Skin Test System or the in vitro Intestinal Test System for Determination of the Penetration of the Pathogen

In order to determine the influence of antimycotics on the penetration of candida albicans in skin and intestinal cells, 12 inserts of the in vitro skin system held in submerse culture and 12 inserts of the in vitro intestinal system each were infected with 10³ pathogenic organisms of the virulent candida strain Sc5314 or the avirulent candida strain Can34. Amphotericin B was added to 5 inserts in a concentration of 0.1, 0.5, 1.0, and 2.0 μg/μl, and fluconazole was added to 5 inserts in a concentration of 0.1, 0.5, 1.0, and 2.0 μg/μl. The inserts were then incubated with shaking at 37° C. for up to 3 days.

The penetration of the pathogen into the organoid structures was investigated after approximately 18 to 24 hours using histological methods on thin sections according to example 4. It was found that the addition of amphotericin B and fluconazole apparently prevents the invasion of the virulent candida strain by inhibiting its growth.

1. Method for preparing a monolayer human or animal in vitro tissue test system, wherein human or animal cells, in a three-dimensional, gel-like biomatrix, containing the cultivated cells and at least 3 mg/ml collagen in buffered, serum-containing cell culture medium, wherein the collagen solution used for the preparation of the biomatrix has a content of non-denatured, native collagen of ≧90% in acidic, aqueous medium, are cultivated in such a way so as to obtain a monolayer in vitro tissue system.

2. Method according to claim 1, wherein the cultivation of the human or animal cells comprises an at least one to two day submerse culture.

3. Method according to claim 1 or 2, wherein the human or animal cells may be non-degenerated cells, degenerated cells, or a mixture thereof.

4. Method according to claim 1 or 2, wherein the human or animal cells may be genetically engineered cells or a mixture of cells not genetically engineered and genetically engineered cells.

5. Method for preparing a monolayer human or animal in vitro tissue test system according to one of claims 1 to 5, comprising the extraction of collagen-containing tissue, the transfer of the collagen-containing tissue into acidic solution, the incubation of the collagen tissue transferred into acidic solution at 2 to 10° C., in particular at 4° C., the centrifuging off of undissolved collagen parts, the mixing of the obtained collagen solution at 2 to 10° C., preferably at 4° C., with a solution containing the human or animal cells to be cultivated, cell culture medium, serum, and buffer, and the gelling of the mixed solution by increasing the temperature.

6. Method according to one of claims 1 to 5, wherein the human or animal cells are, after cultivation in the three-dimensional, gel-like biomatrix, removed from the latter and are further cultivated in a higher cell density so that a monolayer in vitro tissue system is obtained.

7. Method for preparing a three-dimensional, multilayer human or animal in vitro organ or tissue test system, wherein cells of a first human or animal tissue type, in a three-dimensional, gel-like biomatrix containing the cells to be cultivated and at least 3 mg/ml collagen in buffered, serum-containing cell culture medium, wherein the collagen solution used for preparing the biomatrix has a content of non-denatured, native collagen of ≧90% in acidic, aqueous medium, undergo at least a one to two day cultivation, and wherein cells of a second human or animal tissue type are then seeded in a cell culture medium on the biomatrix and are then further cultivated, and wherein, in an optional further step, cells of further human or animal tissue types are then potentially seeded in a cell culture medium and are then further cultivated so that a three-dimensional, multilayer in vitro organ or tissue test system is obtained.

8. Method according to claim 7, wherein the cultivation of the cells of the first human or animal tissue type comprises at least a one to two day submerse culture.

9. Method according to claim 7 or 8, wherein the cultivation of the cells of the second human or animal tissue type comprises at least a 2- to 6-day submerse culture and at least a 10- to 14-day airlift culture.

10. Method according to one of claims 7 to 9, wherein the cells of the second human or animal tissue type and potentially the cells of the further human or animal tissue types have a high content of undifferentiated stem cells and/or immune system cells.

11. Method according to one of claims 7 to 10, wherein the cells of the first human or animal tissue type and the cells of the second human or animal tissue type as well as the cells of any further human or animal tissue types potentially used may be non-degenerated, degenerated cells or a mixture thereof.

12. Method according to one of claims 7 to 10, wherein the cells of the first human or animal tissue type and the cells of the second human or animal tissue type as well as the cells of any further human or animal tissue types potentially used may be genetically engineered cells or a mixture of cells not genetically engineered and genetically engineered cells.

13. Method according to one of claims 7 to 12, wherein the cells of the first human or animal tissue type are dermal fibroblasts and the cells of the second human or animal tissue type are keratinocytes so that after the cultivation a three-dimensional in vitro skin test system is prepared.

14. Method according to one of claims 7 to 12, wherein the cells of the first human or animal tissue type are connective tissue cells of the intestine and the cells of the second human or animal tissue type are enterocytes or intestional epithelial cells so that after the cultivation a three-dimensional in vitro intestinal test system is prepared.

15. Method according to one of claims 7 to 12, wherein the cells of the first human or animal tissue type are corneal endothelial cells, the cells of the second human or animal tissue type are corneal keratinocytes, and the cells of a further, third human or animal tissue type are corneal epithelial cells so that after cultivation a three-dimensional in vitro corneal test system is prepared.

16. Methdo according to one of claims 7 to 12, whereby the cells of the first human or animal tissue type are fibroblasts, in particular tracheal fibroblasts, and the cells of the second human or animal tissue type are tracheal epithelial cells to that after cultivation a three-dimensional in vitro trachea test system is prepared.

17. Method according to one of claims 7 to 12, wherein the cells of the first human or animal tissue type are fibroblasts, in particular mucosal fibroblasts, and the cells of the second human or animal tissue type are mucosal epithelial cells so that after cultivation a three-dimensional in vitro mucosa test system is prepared.

18. Method for preparing a three-dimensional multilayer in vitro organ or tissue test system according to one of claims 7 to 17, comprising the extraction of collagen-containing tissue, the transfer of the collagen-containing tissue into acidic solution, the incubation of the collagen tissue transferred into acidic solution at 2 to 10° C., in particular at 4° C., the centrifuging off of undissolved collagen parts, the mixing of the obtained collagen solution at 2 to 10° C., preferably at 4° C., with a solution containing the cells to be cultivated of a first human or animal cell type, cell culture medium, serum, and buffer, the gelling of the mixed solution by increasing the temperature, the incubation of the gelled mixture at 37° C., and the seeding of the cells of the second human or animal tissue system, and potentially the seeding of the cells of the second human or animal tissue types onto the incubated, gelled mixture.

19. Method according to one of claims 1 to 18, wherein the biomatrix containing the embedded human or animal cells is prepared in that collagen fibers, extracted from a tissue, are stirred in acidic solution for 3 to 14 days at 2 to 10° C., preferably at 4° C., the undissolved collagen parts are centrifuged off, and the resulting finished collagen solution with a collagen content of 3 mg/ml to 8 mg/ml is mixed with a solution containing the human or animal cells to be embedded, cell culture medium, serum, and buffer at 2 to 10° C., preferably at 4° C., and is then gelled at a higher temperature, preferably at room temperature up to 37° C.

20. Method according to claim 19, wherein the acidic solution is acetic acid solution, in particular a 0.1% acetic acid solution.

21. Method according to claim 19 or 20, wherein the solution containing the cells, cell culture medium, serum, and buffer is mixed with a volume ratio of 1:1 with the collagen-containing solution.

22. Monolayer in vitro tissue test system prepared according to a method according to one of claims 1 to 6 or 19 to 21.

23. Three-dimensional, multilayer in vitro skin test system, prepared according to a method according to one of claims 7 to 12 or 18 to 21.

24. Three-dimensional, multi-layer in vitro intestinal test system, prepared in accordance with a method according to one of claims 7 to 12, 14, or 18 to 21.

25. Three-dimensional, multilayer in vitro corneal test system, prepared according to a method according to one of claims 7 to 12, 15, or 18 to 21.

26. Three-dimensional, multilayer in vitro tracheal test system, prepared according to a method according to one of claims 7 to 12, 16, or 18 to 21.

27. Three-dimensional, multilayer in vitro mucosal test system, prepared according to a method according to one of claims 7 to 12 or 17 to 21.

28. Method for studying the infection process of a pathogenic or parasitic microorganism in a human or animal tissue, comprising the bringing into contact of a monolayer in vitro tissue test system according to claim 22 or a three-dimensional multilayer in vitro organ or tissue test system according to one of claims 23 to 27 with a pathogenic or parasitic microorganism, and the co-cultivation of the microorganism with the mono- or multilayer in vitro organ or tissue test system under conditions that permit the subsequenct or simultaneous study of the infection process.

29. Method according to claim 28, wherein the co-cultivation takes place in a submerse culture, and the adhesion of the microorganism to cells of the mono- or multilayer in vitro organ or tissue test system is determined.

30. Method according to claim 28, wherein the co-cultivation takes place in an airlift culture, and the penetration of the microorganism into cells of the mono- or multilayer in vitro organ or tissue test system is determined.

31. Method for determining the effect of a chemical substance or of an agent on the infection process of a pathogenic or parasitic microorganism in a human or animal tissue, comprising the bringing into contact of a monolayer in vitro tissue test system according to claim 22 or a three-dimensional in vitro organ or tissue test system according to one of claims 23 to 27 with a pathogenic or parasitic microorganism, the co-cultivation of the microorganism with the mono- or multilayer in vitro organ or tissue test system in the presence or absence of the studied substance or agent, and the determination of the interaction between the mono- or multilayer in vitro organ or tissue test system, the micoorganism, and the substance to be tested or the agent to be tested.

32. Method according to claim 31, wherein the influence of the substance to be tested or the agent to be tested on the adhesion of the microorganism is determined.

33. Method according to claim 31, wherein the influcence of the substance to be tested or the agent to be tested on the penetration of the microorganism is determined.

34. Method according to claim 31, wherein the influence of the substance to be tested or the agent to be tested on the growth rate of the microorganism is determined.

35. Method for determining the effect of a chemical substance or of an agent on non-degenerated or degenerated human or animal cells, comprising the bringing into contact of the chemical substance or of the agent with a monolayer in vitro tissue test system according to claim 22 constructed of degenerated and/or non-degenerated human or animal cells or a three-dimensional in vitro organ or tissue test system according to claims 23 to 27 constructed of degenerated and/or non-degenerated human or animal cells, and the determination of the interaction between the mono- or multilayer in vitro organ or tissue test system, the microorganism and the chemical substance or agent.
